SEQUENCE LISTING (42 sequences)

SEQ ID NO: 1 - RNA, 25 nt, Homo sapiens
ccucaagaaugauggcaccuucauu

SEQ ID NO: 2 - RNA, 25 nt, Homo sapiens
cacugucaucgaacgcaccuuccau

SEQ ID NO: 3 - RNA, 25 nt, Homo sapiens
ucaucgaacgcaccuuccaugugga

SEQ ID NO: 4 - RNA, 25 nt, Homo sapiens
gcgugaccaugaacgaguuugagua

SEQ ID NO: 5 - RNA, 25 nt, Homo sapiens
ccaugaacgaguuugaguaccugaa

SEQ ID NO: 6 - RNA, 25 nt, Homo sapiens
ugcuggacaaggacgggcacauuaa

SEQ ID NO: 7 - RNA, 25 nt, Homo sapiens
aguccuugcuuucagggcugcucaa

SEQ ID NO: 8 - RNA, 25 nt, Homo sapiens
cagcacguguacgagaagaagcuca

SEQ ID NO: 9 - RNA, 25 nt, Homo sapiens
ccugaccaagaugacagcauggagu

SEQ ID NO: 10 - RNA, 25 nt, Homo sapiens
acgucggagacugacaccagguauu

SEQ ID NO: 11 - DNA, 25 bp, Homo sapiens
ggaaugaaccuucugcaacaucuta

SEQ ID NO: 12 - DNA, 25 bp, Homo sapiens
auucaguggcuuaaucaugaaugtc

SEQ ID NO: 13 - DNA, 25 bp, Homo sapiens
cuugaauuuaaucauguuguuucta

SEQ ID NO: 14 - DNA, 25 bp, Homo sapiens
cagcuaaaggugaagauauauucct

SEQ ID NO: 15 - DNA, 25 bp, Homo sapiens
gugcagauaaugacaaggaauauct

SEQ ID NO: 16 - RNA, 25 nt, Homo sapiens
aauggaaacaagaauauucagucca

SEQ ID NO: 17 - DNA, 25 bp, Homo sapiens
aaaugugcaguguugaaucauuuct

SEQ ID NO: 18 - RNA, 25 nt, Homo sapiens
auggaaacaagaauauucaguccaa

SEQ ID NO: 19 - DNA, 25 bp, Homo sapiens
cuauuguaaagcuaaugugaagata

SEQ ID NO: 20 - DNA, 25 bp, Homo sapiens
acaagaauauucaguccaaacugtt

SEQ ID NO: 21 - RNA, 25 nt, Homo sapiens
aagaugauguuugaaacuauuccaa

SEQ ID NO: 22 - DNA, 25 bp, Homo sapiens
ggcuuaaucaugaaugucuaaauat

SEQ ID NO: 23 - RNA, 25 nt, Homo sapiens
aucugcaccaagcaugaacaauugg

SEQ ID NO: 24 - DNA, 25 bp, Homo sapiens
ccugaaagcagguaaacuuaaccta

SEQ ID NO: 25 - DNA, 25 bp, Homo sapiens
gaaucugcaccaagcaugaacaatt

SEQ ID NO: 26 - DNA, 25 bp, Homo sapiens
aaucugcaccaagcaugaacaautg

SEQ ID NO: 27 - DNA, 25 bp, Homo sapiens
cugaaagcagguaaacuuaaccuac

SEQ ID NO: 28 - RNA, 25 nt, Homo sapiens
cacaccugaaagcagguaaacuuaa

SEQ ID NO: 29 - DNA, 25 bp, Homo sapiens
aggacaguaaauauuuuagauguta

SEQ ID NO: 30 - RNA, 25 nt, Homo sapiens
gugaaucugcaccaagcaugaacaa

SEQ ID NO: 31 - DNA, 25 bp, Homo sapiens
augaagaggaagaaucaaaaauctt

SEQ ID NO: 32 - RNA, 25 nt, Homo sapiens
ggacaguaaauauuuuagauguuaa

SEQ ID NO: 33 - DNA, 25 bp, Homo sapiens
ggcguggucauguacgagaugcugt

SEQ ID NO: 34 - RNA, 25 nt, Homo sapiens
aaggaggucauuguggccaaggacg

SEQ ID NO: 35 - DNA, 25 bp, Homo sapiens
gucauuguggccaaggacgaggutg

SEQ ID NO: 36 - RNA, 25 nt, Homo sapiens
guuuuguuaaucuuagugacaagaa

SEQ ID NO: 37 - RNA, 24 nt, Homo sapiens
ggaggucauuguggccaaggacga

SEQ ID NO: 38 - RNA, 25 nt, Homo sapiens
gggguuuuguuaaucuuagugacaa

SEQ ID NO: 39 - RNA, 25 nt, Homo sapiens
gguuuuguuaaucuuagugacaaga

SEQ ID NO: 40 - RNA, 25 nt, Homo sapiens
gaagaaggaggucauuguggccaag

SEQ ID NO: 41 - RNA, 25 nt, Homo sapiens
guuaaucuuagugacaagaaagccc

SEQ ID NO: 42 - DNA, 25 bp, Homo sapiens
gcguggucauguacgagaugcugtg

(19) United States
(12) Patent Application Publication
(10) Pub. No.: US 2021/0222174 A1
(43) Pub. Date: Jul. 22, 2021

(54) COMPOSITIONS AND METHODS FOR THE TREATMENT OF ANESTHESIA-INDUCED NEUROTOXICITY

(71) Applicant: John Mansell, Gillette, WY (US)

(72) Inventor: John Mansell, Gillette, WY (US)

(21) Appl. No.: 17/255,051

(22) PCT Filed: Jun. 28, 2019
(86) PCT No.: PCT/US2019/039986

§ 371 Date: Dec. 22, 2020

Related U.S. Application Data
(60) Provisional application No. 62/692,460, filed on Jun. 29, 2018.

(51) Int. Cl. C12N 15/113; A61K 9/51; A61K 9/127; A61K 9/06

(52) U.S. Cl. CPC C12N 15/113; A61K 9/5184; C12N 2310/11; A61K 9/06; C12N 2310/14; A61K 9/127

(57) ABSTRACT

A formulation comprising an oligonucleotide selected from the group consisting of an oligonucleotide having one of SEQ ID No.:1 through SEQ ID No.:42 or a variant thereof. Also, a method of treating anesthesia-induced neurotoxicity. The method may comprise administering the formulation. The formulation may be administered prior to, concomitant with, subsequent to, or combinations thereof administration of a general anesthetic comprising a fluorinated compound. The oligonucleotide may be incorporated into a carrier system, for example, a liposome, a biodegradable polymer, a hydrogel, or a cyclodextrin, a nucleic acid complex, a virosome, or combinations thereof. Also, a method of treating anesthesia-induced neurotoxicity.

### CROSS-REFERENCE TO RELATED APPLICATIONS

This application claims priority to U.S. Provisional Application Ser. No. 62/692,460, filed Jun. 29, 2018, by Dr. John Mansell and titled “Compositions and Methods for the Treatment of Anesthesia-Induced Neurotoxicity” which is incorporated herein by reference in its entirety.

### REFERENCE TO SEQUENCE LISTING

The content of the ASCII text file of the sequence listing named “4983_01300_Sequence_Listing.txt” which is 6,380 bytes in size and was created on Jun. 18, 2018 using PatentIn version 3.5 and electronically submitted via the USPTO's “EFS-Web” patent application and document submission system herewith is incorporated herein by reference in its entirety. The incorporated sequence listing comprises SEQ ID No.:1 through SEQ ID No.:42.

### TECHNICAL FIELD

The present disclosure generally relates to compositions and methodologies for the treatment of anesthesia-induced neurotoxicity. More specifically this disclosure relates to prophylactic and/or therapeutic utilization of oligonucleotides for the treatment of anesthesia-induced neurotoxicity in pediatric subjects.

### BACKGROUND

Each year, about six million children, including 1.5 million infants, in the United States undergo surgery with general anesthesia, often requiring repeated exposures. General anesthesia encompasses the administration of agents that induce analgesic, sedative, and muscle relaxant effects. Although the mechanisms of action of general anesthetics are still not completely understood, recent data have suggested that anesthetics primarily modulate two major neurotransmitter receptor groups, either by inhibiting N-methyl-D-aspartate (NMDA) receptors, or conversely by activating 7-aminobutyric acid (GABA) receptors. In developing brains, which are more sensitive to disruptions in activity-dependent plasticity, this transient inhibition may have long term neurodevelopmental consequences. Accumulating reports from preclinical studies show that anesthetics in neonates cause cellular toxicity including apoptosis and neurodegeneration in the developing brain. Importantly, animal and clinical studies indicate that exposure to general anesthetics may affect CNS development, resulting in long-lasting cognitive and behavioral deficiencies, such as learning and memory deficits, as well as abnormalities in social memory and social activity.

Gene expression analysis has suggested the increased expression of autophagy promoting proteins as one potential cause for the observed anesthesia-induced neurotoxicity. For example, dysregulated signaling in the Ras/PI3K/PTEN/Akt/mTOR pathway often results in increased sensitivity to apoptotic-inducing agents. An ongoing need exists for methods and compositions to alleviate anesthesia-induced neurotoxicity (AIN).

### SUMMARY

In some embodiments is a formulation comprising an oligonucleotide selected from the group consisting of an oligonucleotide having one of SEQ ID No.:1 through SEQ ID No.:42 or a variant thereof. For example, the oligonucleotide may comprise at least 75% sequence identity to one of SEQ ID No.:1 through SEQ ID No.:42, or at least 85% sequence identity to one of SEQ ID No.:1 through SEQ ID No.:42, or at least 95% sequence identity to one of SEQ ID No.:1 through SEQ ID No.:42, or comprises one of SEQ ID No.:1 through SEQ ID No.:42. In some embodiments, the oligonucleotide may be incorporated into a carrier system, for example, a liposome, a biodegradable polymer, a hydrogel, or a cyclodextrin, a nucleic acid complex, a virosome, or combinations thereof.

Additionally, in some embodiments is a method of treating anesthesia-induced neurotoxicity. The method may comprise administering a formulation comprising an oligonucleotide selected from the group consisting of an oligonucleotide having one of SEQ ID No.:1 through SEQ ID No.:42 or a variant thereof to a subject. For example, the oligonucleotide may comprise at least 75% sequence identity to one of SEQ ID No.:1 through SEQ ID No.:42, or at least 85% sequence identity to one of SEQ ID No.:1 through SEQ ID No.:42, or at least 95% sequence identity to one of SEQ ID No.:1 through SEQ ID No.:42, or comprises one of SEQ ID No.:1 through SEQ ID No.:42. The formulation may be administered prior to, concomitant with, subsequent to, or combinations thereof administration of a general anesthetic comprising a fluorinated compound. In some embodiments, the oligonucleotide may be incorporated into a carrier system, for example, a liposome, a biodegradable polymer, a hydrogel, or a cyclodextrin, a nucleic acid complex, a virosome, or combinations thereof.

### BRIEF DESCRIPTION OF DRAWING

FIG. 1 illustrates a schematic of the Ras/PI3K/PTEN/Akt/mTOR pathway.

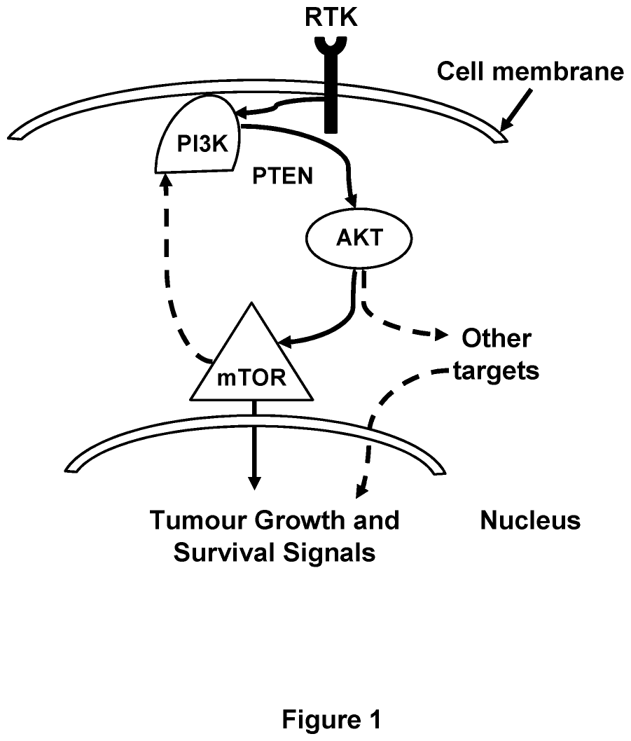

### DETAILED DESCRIPTION

Disclosed herein are methods of treating AIN. In an aspect, the AIN is the result of exposure to anesthesia, typically general anesthesia. In an aspect the anesthesia is an inhaled anesthetic. In an aspect the anesthesia is an intravenous anesthetic. For example, and without limitation, the anesthesia comprises isoflurane, sevoflurane, halothane or combinations thereof.

The terms “treat,” “treating,” or “treatment,” as used herein, include alleviating, abating, or ameliorating a disease or condition, or symptoms thereof; managing a disease or condition, or symptoms thereof, preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a symptom caused by the disease or condition; and/or stopping the symptoms of the disease or condition. Treatment as used herein also encompasses any pharmaceutical or medicinal use of the compositions herein.

The term “subject” as used herein, refers to an animal which is the object of treatment, observation, or experiment. By way of example only, a subject may be, but is not limited to, a mammal including, but not limited to, a human. In an aspect the subject is a pediatric patient to be administered an inhaled anesthetic by a healthcare professional.

In an aspect, the subject is administered the compositions disclosed herein in a therapeutically effective amount sufficient for treating, preventing, and/or ameliorating one or more symptoms of AIN. As used herein, amelioration of the symptoms of AIN by administration of a particular composition of the type disclosed herein refers to any lessening, whether lasting or transient, which can be attributed to or associated with administration of compositions of the type disclosed herein. It is contemplated that the therapeutically effective amount may be optimized by one or more healthcare professionals in consideration of the particular factors affecting a subject.

As used herein, the term “RNA interference” or “RNAi” refers to the silencing or decreasing of gene expression by iRNA agents (e.g., siRNAs, miRNAs, shRNAs), via the process of sequence-specific, post-transcriptional gene silencing in animals and plants, initiated by an iRNA agent that has a seed region sequence in the iRNA guide strand that is complementary to a sequence of the silenced gene. As used herein, the term an “iRNA agent” (abbreviation for “interfering RNA agent”), refers to an RNA agent, or chemically modified RNA, which can down-regulate the expression of a target gene. The phrase “chemical modification” as used herein refers to its meaning as is generally accepted in the art. With reference to exemplary nucleic acid molecules of the present disclosure, the term refers to any modifications of the chemical structure of the nucleotides that differs from nucleotides of native siRNA or RNA in general. The term “chemical modification” encompasses the addition, substitution, or modification of native siRNA or RNA at the sugar, base, or internucleotide linkage, as described herein or as is otherwise known in the art. In certain aspects, the term “chemical modification” can refer to certain forms of RNA that are naturally occurring in certain biological systems, for example 2′-O-methyl modifications or inosine modifications. While not wishing to be bound by theory, an iRNA agent may act by one or more of a number of mechanisms, including post-transcriptional cleavage of a target mRNA, or pre-transcriptional or pre-translational mechanisms. An iRNA agent can include a single strand (ss) or can include more than one strands, e.g. it can be a double stranded (ds) IRNA agent. As used herein, the term “siRNA” refers to a small interfering RNA. siRNAs include short interfering RNA of about 15-60, 15-50, or 15-40 (duplex) nucleotides in length, more typically about 15-30, 15-25 or 19-25 (duplex) nucleotides in length, and is alternatively about 20-24 or about 21-22 or 21-23 (duplex) nucleotides in length (e.g., each complementary sequence of the double stranded siRNA is 15-60, 15-50, 15-40, 15-30, 15-25 or 19-25 nucleotides in length, alternatively about 20-24 or about 21-22, or 21-23 nucleotides in length, alternatively 19-21 nucleotides in length, and the double stranded siRNA is about 15-60, 15-50, 15-40, 15-30, 15-25 or 19-25, alternatively about 20-24, or about 21-22 or 19-21 or 21-23 base pairs in length). siRNA duplexes may comprise 3′ overhangs of about 1 to about 4 nucleotides, alternatively about 2 to 3 nucleotides and 5′ phosphate termini. In some aspects, the siRNA lacks a terminal phosphate. In some aspects, one or both ends of siRNAs can include single-stranded 3′ overhangs that are two or three nucleotides in length, such as, for example, deoxythymidine (dTdT) or uracil (UU) that are not complementary to the target sequence. In some aspects, siRNA molecules can include nucleotide analogs (e.g., thiophosphate or G-clamp nucleotide analogs), alternative base linkages (e.g., phosphorothioate, phosphonoacetate, or thiophosphonoacetate) and other modifications useful for enhanced nuclease resistance, enhanced duplex stability, enhanced cellular uptake, or cell targeting.

In an aspect, the oligonucleotides disclosed herein are used to treat pediatric AIN and thus are designated PAIN. As used herein, the PAINs need not be limited to those molecules containing only RNA but may further encompass chemically-modified nucleotides and non-nucleotides. In certain aspects, the PAINs of the present disclosure comprise separate sense and antisense sequences or regions, wherein the sense and antisense regions are covalently linked by nucleotide or non-nucleotide linkers molecules as is known in the art, or are alternately non-covalently linked by ionic interactions, hydrogen bonding, Van der waals interactions, hydrophobic interactions, and/or stacking interactions. In certain aspects, the PAINs of the present disclosure comprise nucleotide sequence that is complementary to nucleotide sequence of a target gene. In another aspect, the PAINs of the present disclosure interact with nucleotide sequence of a target gene in a manner that causes inhibition of expression of the target gene.

As used herein, “percent modification” refers to the number of nucleotides in the PAIN (e.g., iRNA, or each of the strand of the siRNA or to the collective dsRNA) that have been modified. For example, a 19% modification of the antisense strand of a PAIN refers to the modification of up to 4 nucleotides/bp in a 21-nucleotide sequence (21 mer). 100% modification refers to a fully modified dsRNA. The extent of chemical modification will depend upon various factors such as for example, target mRNA, off-target silencing, degree of endonuclease degradation, etc.

As used herein, the term “shRNA” or “short hairpin RNAs” refers to individual transcripts that adopt stem-loop structures which are processed into siRNA by RNAi machinery. Typical shRNA molecules comprise two inverted repeats containing the sense and antisense target sequence separated by a loop sequence. The base-paired segment may vary from 17 to 29 nucleotides, wherein one strand of the base-paired stem is complementary to the mRNA of a target gene. The loop of the shRNA stem-loop structure may be any suitable length that allows inactivation of the target gene in vivo. While the loop may be from 3 to 30 nucleotides in length, typically it is 1-10 nucleotides in length. The base paired stem may be perfectly base paired or may have 1 or 2 mismatched base pairs. The duplex portion may, but typically does not, contain one or more bulges consisting of one or more unpaired nucleotides. The shRNA may have non-base-paired 5′ and 3′ sequences extending from the base-paired stem. Typically, however, there is no 5′ extension. The first nucleotide of the shRNA at the 5′ end is a G, because this is the first nucleotide transcribed by polymerase III. If G is not present as the first base in the target sequence, a G may be added before the specific target sequence. The 5′ G typically forms a portion of the base-paired stem. Typically, the 3′ end of the shRNA is a poly U segment that is a transcription termination signal and does not form a base-paired structure. As described in the application and known to one skilled in the art, shRNAs are processed into siRNAs by the conserved cellular RNAi machinery. Thus, shRNAs are precursors of siRNAs and are, in general, similarly capable of inhibiting expression of a target mRNA transcript.

As used herein, the term “isolated” in the context of an isolated nucleic acid molecule (e.g., PAIN), is one which is altered or removed from the natural state through human intervention. For example, an RNA naturally present in a living animal is not “isolated.” A synthetic RNA or dsRNA or microRNA molecule partially or completely separated from the coexisting materials of its natural state, is “isolated.”

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “gene” refers to a nucleic acid (e.g., DNA or RNA) sequence that comprises coding sequences necessary for the production of an RNA and/or a polypeptide, or its precursor as well as noncoding sequences (untranslated regions) surrounding the 5′ and 3′ ends of the coding sequences. The term “gene” encompasses both cDNA and genomic forms of a gene. A functional polypeptide can be encoded by a full-length coding sequence or by any portion of the coding sequence as long as the desired activity or functional properties (e.g., enzymatic activity, ligand binding, signal transduction, antigenic presentation) of the polypeptide are retained. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

As used herein the term “substantial silencing” means that the mRNA of the targeted gene (e.g., PTEN) is inhibited and/or degraded by the presence of the introduced PAIN, such that expression of the targeted gene is reduced by about 10% to 100% as compared to the level of expression seen when the PAIN is not present. Generally, when a gene is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction in expression as compared to when the PAIN is not present. As used herein the term “substantially normal activity” means the level of expression of a gene when a PAIN has not been introduced. As used herein the terms “inhibit,” “down-regulate,” or “reduce” as used herein refers to its meaning as is generally accepted in the art. With reference to exemplary nucleic acid molecules of the present disclosure, the term generally refers the reduction in the expression of the gene, or level of RNA molecules or equivalent RNA molecules encoding one or more proteins or protein subunits, or activity of one or more proteins or protein subunits, below that observed in the absence of the nucleic acid molecules (e.g., PAIN) of the present disclosure. Down-regulation can also be associated with post-transcriptional silencing, such as, RNAi mediated cleavage or by alteration in DNA methylation patterns or DNA chromatin structure. Inhibition, down-regulation or reduction with a PAIN can be in reference to an inactive molecule, an attenuated molecule, an oligonucleotide with a scrambled sequence, or an oligonucleotide with mismatches or alternatively, it can be in reference to the system in the absence of the oligonucleotide.

In an aspect, the compositions disclosed herein comprise a PAIN which results in a down-regulation or reduction in the expression of a phosphatidylinositol-3,4,5-trisphosphate 3-encoded by PTEN. In an alternative aspect, the compositions disclosed herein comprise a PAIN which results in a down-regulation or reduction in the a RAC-alpha serine/threonine-protein kinase (Protein Kinase B or PKB) encoded by AKT1. In an alternative aspect, the compositions disclosed herein comprise a PAIN which results in a down-regulation or reduction in the C/EBP homologous protein (CHOP) encoded by DDIT3.

In another aspect, the PAIN comprises an oligonucleotide that inhibits the expression of AKT1 or alternatively substantially silences the expression AKT1. In yet another aspect, the PAIN comprises an oligonucleotide that inhibits expression of DDIT3 or alternatively substantially silences the expression of DDIT3.

In an aspect, the PAIN comprises an oligonucleotide that inhibits expression of the gene coding for the PTEN protein or alternatively substantially silences the expression of the gene coding for the PTEN protein. The phosphatase and tensin homologue (PTEN) is essential for normal cell maintenance and is well characterized as a key tumor suppressor. PTEN is pivotal in the regulation of the receptor tyrosine kinase (RTK) PI-3 kinase (PI3K)/Akt signaling pathway and, as such, even small changes in PTEN expression have been shown to have major consequences for normal cellular function. The PTEN protein translocates between the nucleus and the cytoplasm enabling PTEN-specific compartmentalized functions. At the molecular level, PTEN expression and cellular abundance is tightly regulated at the transcriptional, post-translational and post-transcriptional levels. The PTEN gene is encoded in 9 exons and has a 1212 nucleotide (nt) open reading frame. The gene encodes a polypeptide of 403 amino acids with a relative molecular mass of 47 kDa. The PTEN protein consists of two major domains, the N-terminal phosphatase catalytic domain (residues 7-185) and a C-terminal domain (residues 186-351). These two domains together form a minimal catalytic unit and comprise almost the entire protein, excluding only a very short N-terminal tail.

In another aspect, the PAIN comprises an oligonucleotide that inhibits the expression of AKT1 or alternatively substantially silences the expression AKT1. The PI3K/Akt pathway has been one of the most intensively investigated signaling networks in cancer research. AKT is hyperactivated in cancer cells by multiple mechanisms, including the loss of PTEN, mutations that activate the catalytic subunit of PI3K, p110α, mutations that activate Akt isoforms, the activation of RAS and growth factor receptors and amplification of the genes encoding the catalytic subunit of PI3K and Akt. AKT1 encodes a 57-kDa serine/threonine kinase, originally identified as an inactivator of glycogen synthase (GSK3β) in response to insulin-like growth factor. PKB, when activated by phosphorylation on amino acids Thr308 and Ser473 by phosphoinositide3-kinase (PI3-kinase), has several important effects (including inhibition of apoptosis by phosphorylation and inactivation of pro-apoptotic factors Bad and caspase-9).

The transcription factor CCAAT-enhancer-binding protein homologous protein (CHOP) was first reported as a molecule involved in endoplasmic reticulum (ER) stress-induced apoptosis. CHOP expression is low under non-stressed conditions, but its expression markedly increases in response to ER stress through IRE1-, PERK- and ATF6-dependent transcriptional induction. The activation of ATF4, which is induced by the PERK-mediated phosphorylation of eIF2α, is thought to play a dominant role in the induction of CHOP in response to ER stress. The overexpression of CHOP promotes apoptosis in several cell lines, whereas CHOP-deficient cells are resistant to ER stress-induced apoptosis. Therefore, CHOP plays an important role in the induction of apoptosis. Two isoforms of CHOP are generated from its mRNA by a ribosomal scanning mechanism (14, 15). The full-length protein is 42 kDa and contains three transactivation domains

The extent of downregulation of PTEN, AKT1, DDIT3 or their respective gene products may be determined using any suitable assay. Suitable assays include without limitation, e.g., examination of protein or mRNA levels using any suitable technique such as dot blots, northern blots, in situ hybridization, ELISA, microarray hybridization, immunoprecipitation, enzyme function, as well as phenotypic assays known to those of skill in the art. To examine the extent of gene silencing, a test sample (e.g., a biological sample from organism of interest expressing the target gene(s) or a sample of cells in culture expressing the target gene(s)) is contacted with a PAIN that silences, reduces, or inhibits expression of the target gene(s). Expression of the target gene in the test sample is compared to expression of the target gene in a control sample (e.g., a biological sample from organism of interest expressing the target gene or a sample of cells in culture expressing the target gene) that is not contacted with the PAIN. Control samples (i.e., samples expressing the target gene) are assigned a value of 100%. In an aspect, substantial silencing, inhibition, down-regulation or reduction of expression of a target gene is achieved when the value of test the test sample relative to the control sample is about 95%, 90%, 85%, 80%, 75%, 70%, 65%, 60%, 55%, 50%, 45%, 40%, 35%, 30%, 25%, 20%, or 10%.

In an aspect the PAIN is a microRNA (miRNA, miR). miRs refer to single-stranded RNA molecules that are generally 21-23 nucleotides in length which regulate gene expression. MicroRNAs are processed from primary transcripts known as pri-miRNA to short stem-loop structures called precursor (pre)-miRNA and finally to functional, mature microRNA. Mature microRNA molecules are partially complementary to one or more messenger RNA molecules, and their primary function is to down-regulate gene expression through the RNAi pathway.

In an aspect, the PAIN is a small interfering RNA (siRNA). Naturally occurring RNAi, a double-stranded RNA (dsRNA) is cleaved by an RNase II/helicase protein, Dicer, into small interfering RNA (siRNA) molecules, a dsRNA of 19-27 nucleotides (nt) with 2-nt overhangs at the 3′ ends. siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it. These and other characteristics of RISC, siRNA molecules, and RNAi have been described.

In an aspect of the present disclosure, the PAIN is an antisense oligonucleotide. Antisense oligonucleotides (ASOs) are synthetic nucleic acids that bind to a complementary target and suppress function of that target. Typically, ASOs are used to reduce or alter expression of RNA targets, particularly messenger RNA (mRNA) or microRNA (miRNA) species. As a general principle, ASOs can suppress gene expression via two different mechanisms of action, including: 1) by steric blocking, wherein the ASO tightly binds the target nucleic acid and inactivates that species, preventing its participation in cellular biology, or 2) by triggering degradation, wherein the ASO binds the target and leads to activation of a cellular nuclease that degrades the targeted nucleic acid species. One class of “target degrading” ASOs are “RNase H active”; formation of heteroduplex nucleic acids by hybridization of the target RNA with a DNA-containing “RNase H active” ASO forms a substrate for the enzyme RNase H. RNase H degrades the RNA portion of the heteroduplex molecule, thereby reducing expression of that species. Degradation of the target RNA releases the ASO, which is not degraded, which is then free to recycle and can bind another RNA target of the same sequence.

In an aspect, a PAIN comprises a microRNA, a siRNA, an ASO, an iRNA, an iRNA agent, an shRNA, a functional variant thereof, or combinations thereof. In some aspects, a functional variant of an oligonucleotide disclosed herein comprises at least 70% sequence identity with any sequence disclosed herein, alternatively at least 75%, alternatively at least 80%, alternatively at least 85%, alternatively at least 90% or alternatively at least 95%. In general, “identity” refers to an exact nucleotide-to-nucleotide correspondence of two oligonucleotides or polynucleotides sequences. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as Wisconsin Sequence Analysis Package, Version 8 (available from Genetics Computer Group, Madison, Wis.) for example, the BESTFIT, FASTA and GAP programs, which rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As identified in the SEQUENCE LISTING below, Sequence ID No. 1 through Sequence ID No. 42 (i.e., <210>1 through <210>42) are representative of the PAINs described herein. In an aspect, the PAIN comprises an oligonucleotide having any one of Sequence ID No. 1 through Sequence ID No. 42, alternatively a functional variant thereof. In some aspects, a PAIN suitable for use in the present disclosure comprises at least 70% sequence identity with any one of Sequence ID No. 1 through Sequence ID No. 42 (i.e., <210>1 through <210>42), or at least 75% sequence identity with any one of Sequence ID No. 1 through Sequence ID No. 42 (i.e., <210>1 through <210>42), or at least 80% sequence identity with any one of Sequence ID No. 1 through Sequence ID No. 42 (i.e., <210>1 through <210>42), or at least 85% sequence identity with any one of Sequence ID No. 1 through Sequence ID No. 42 (i.e., <210>1 through <210>42), alternatively at least 90% sequence identity with any one of Sequence ID No. 1 through Sequence ID No. 42 (i.e., <210>1 through <210>42), or at least 95% sequence identity with any one of Sequence ID No. 1 through Sequence ID No. 42 (i.e., <210>1 through <210>42).

In an aspect, the PAIN has from about 20% to about a 90% modification or alternatively from about a 40% to about 60% modification.

In an aspect, PAINs of the present disclosure (modified or unmodified) are chemically synthesized. Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng. 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end.

Alternatively, PAIN of the present disclosure that interact with and down-regulate PTEN, AKT1 or DDIT3 can be expressed and delivered from a transcript inserted into DNA or RNA vectors. The recombinant vectors can be DNA plasmids or viral vectors. Nonlimiting examples of PAIN expressing viral vectors can be constructed based on adeno-associated virus, retrovirus, adenovirus, or alphavirus.

In some aspects, pol III based constructs are used to express PAINs of the present disclosure. Transcription of the siNA molecule sequences can be driven from a promoter for eukaryotic RNA polymerase I (pol I), RNA polymerase II (pol II), or RNA polymerase III (pol III), (see for example, Thompson, U.S. Pat. Nos. 5,902,880 and 6,146,886). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters may also be used, providing that the prokaryotic RNA polymerase enzyme is expressed in the appropriate cells These exemplary transcription units can be incorporated into a variety of vectors for introduction into mammalian cells, including but not restricted to, plasmid DNA vectors, viral DNA vectors (such as adenovirus or adeno-associated virus vectors). or viral RNA vectors (such as retroviral or alphavirus vectors).

Vectors used to express the PAINs of the present disclosure can encode one or both strands of an siNA duplex, or a single self-complementary strand that self hybridizes into an siRNA duplex. The nucleic acid sequences encoding the PAINs of the present disclosure can be operably linked in a manner that allows expression of the PAIN. In some aspects, the constructs comprising PAINs may additionally comprise reporter genes (e.g., green fluorescent protein) and selection genes (e.g., for antibiotic resistance).

In an alternative aspect, the PAINs of the present are added directly, or can be complexed with cationic lipids, packaged within liposomes, or as a recombinant plasmid or viral vectors which express the PAIN, or otherwise delivered to target cells or tissues. Nucleic acid molecules can be administered to cells by any suitable methodology, including, but not restricted to, encapsulation in liposomes, by iontophoresis, or by incorporation into other vehicles, such as biodegradable polymers, hydrogels, cyclodextrins poly(lactic-co-glycolic)acid (PLGA) and PLCA microspheres, biodegradable nanocapsules, and bioadhesive microspheres, or by proteinaceous vectors. In one aspect, the present disclosure provides carrier systems containing the PAINs described herein. In some aspects, the carrier system is a lipid-based carrier system, cationic lipid, or liposome, nucleic acid complexes, a liposome, a micelle, a virosome, a lipid nanoparticle or a mixture thereof. In other aspects, the carrier system is a polymer-based carrier system such as a cationic polymer-nucleic acid complex. In additional aspects, the carrier system is a cyclodextrin-based carrier system such as a cyclodextrin polymer-nucleic acid complex. In further aspects, the carrier system is a protein-based carrier system such as a cationic peptide-nucleic acid complex.

In other aspects, the PAIN is a component of a conjugate or complex provided that can impart therapeutic activity by transferring therapeutic compounds across cellular membranes, altering the pharmacokinetics, and/or modulating the localization of nucleic acid molecules of the present disclosure. For example, the conjugate can comprise polyethylene glycol (PEG) can be covalently attached to a PAIN. The attached PEG can be any molecular weight, for example from about 100 to about 50,000 daltons (Da).

In yet other aspects, the PAIN is a component of compositions or formulations comprising surface-modified liposomes containing poly (ethylene glycol) lipids (PEG-modified, or long-circulating liposomes, or stealth liposomes) and PAINs. In some aspects, the siRNA molecules of the present disclosure can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In an aspect, the PAINs of this disclosure are prepared into a composition or formulation for administration to a subject. The terms “composition” or “formulation” as used herein refer to their generally accepted meaning in the art. These terms generally refer to a composition or formulation, such as in a pharmaceutically acceptable carrier or diluent, in a form suitable for administration, e.g., systemic or local administration, into a cell or subject, including, for example, a human. Suitable forms, in part, depend upon the use or the route of entry, for example oral, transdermal, inhalation, or by intravenous, intramuscular or intrathecal injection. Such forms should not prevent the composition or formulation from reaching a target cell (i.e., a cell to which the negatively charged nucleic acid is desirable for delivers). For example, compositions injected into the blood stream should be soluble. Other factors include considerations such as toxicity and forms that prevent the composition or formulation from exerting its effect. Non-limiting examples of agents suitable for formulation with the nucleic acid molecules of the instant present disclosure include: Lipid Nanoparticles (see for example Semple et al., 2010, Nat Biotechnol., February; 28(2):172-6); P-glycoprotein inhibitors (such as Pluronic P85); biodegradable polymers, such as poly (DL-lactide-coglycolide) microspheres for sustained release delivery (Emerich, D F et al, 1990, Cell Transplant, 8, 47-58); and loaded nanoparticles, such as those made of polybutylcyanoacrylate. Other non-limiting examples of delivers strategies for the nucleic acid molecules of the instant present disclosure include material described in Boado et al., 1998, J. Pharm. Sci., 87, 1308-1315; Tyler et al., 1999, FEBS Lett., 421, 280-284; Partridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. A “pharmaceutically acceptable composition” or “pharmaceutically acceptable formulation” can refer to a composition or formulation that allows for the effective distribution of the nucleic acid molecules of the instant disclosure to the physical location most suitable for their desired activity.

In an aspect, the formulation may contain additional ingredients. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

In an alternative aspect, the subject is administered a pharmaceutical formulation comprising a PAIN having a sequence as disclosed herein prior to, concomitant with, subsequent to a surgical procedure where the subject was administered a general anesthetic. In such aspects the general anesthetic may comprise a halogenated gaseous compound such as isoflurane or sevoflurane.

Without wishing to be limited by theory, these different forms of oligonucleotides would diminish efficient transcription of PTEN protein, Protein Kinase B or CHOP, reduce successful movement of guide strand mRNA to translation and interfere with efficient translation of mRNA which produces PTEN protein, Protein Kinase B or CHOP.

Conventional methods, known to those of ordinary skill in the art of medicine, can be used to administer the pharmaceutical formulations to a mammalian subject. The pharmaceutical formulations can be administered via oral, subcutaneous, intrapulmonary, transmucosal, intraperitoneal, intrauterine, sublingual, intrathecal or intramuscular routes.

Injectable formulations of the PAIN compositions or formulations of the present disclosure may contain various carriers. Physiologically acceptable excipients may include, for example, 5% dextrose, 0.9% saline, Ringer's solution, or other suitable excipients. Intramuscular preparations, e.g., a sterile formulation of the compounds of the present disclosure can be dissolved and administered in a pharmaceutical excipient was as water-for-injection, 0.9% saline, or 5% glucose solution.

In some aspects, this the formulations disclosed herein would be administered so as to be present in neural and other affected tissues, before, during, and for a period of time after, exposure to anesthetics so as to decrease or prevent anesthetic-triggered apoptotic events. For example, the formulations disclosed herein could be administered at least about 30 minutes prior to exposure to anesthetics, or at least about 1 hour, or at least about 2 hours, or at least about 4 hours, or at least about 8 hours, or at least about 16 hours, or at least about 24 hours, or at least about 32 hours, or at least about 48 hours, or at least about 60 hours, or at least about 72 hours prior to exposure to anesthetics and, additionally or alternatively, substantially contemporaneously with the exposure anesthetics and, additionally or alternatively, about 30 minutes following exposure to anesthetics, or about 1 hour, or about 2 hours, or about 4 hours, or about 8 hours, or about 16 hours, or about 24 hours, or about 32 hours, or at about 48 hours, or about 60 hours, or about 72 hours following exposure to anesthetics.

The following particular aspects are given as particularized aspects of the present disclosure and to demonstrate the practice and advantages thereof. It is understood that the particularized aspects are given by way of illustration and are not intended to limit the specification or the claims to follow in any manner.

Embodiment No. 1 is a formulation comprising an oligonucleotide selected from the group consisting of an oligonucleotide having one of SEQ ID No.:1 through SEQ ID No.:42 or a variant thereof.

Embodiment No. 2 is the formulation of Embodiment No. 1, wherein the oligonucleotide comprises at least 75% sequence identity to one of SEQ ID No.:1 through SEQ ID No.:42.

Embodiment No. 3 is the formulation of one of Embodiment Nos. 1-2, wherein the oligonucleotide comprises at least 85% sequence identity to one of SEQ ID No.:1 through SEQ ID No.:42.

Embodiment No. 4 is the formulation of one of Embodiment Nos. 1-3, wherein the oligonucleotide comprises at least 95% sequence identity to one of SEQ ID No.:1 through SEQ ID No.:42.

Embodiment No. 5 is the formulation of one of Embodiment Nos. 1-4, wherein the oligonucleotide comprises one of SEQ ID No.:1 through SEQ ID No.:42.

Embodiment No. 6 is the formulation of one of Embodiment Nos. 1-5, wherein the oligonucleotide is incorporated into a carrier system.

Embodiment No. 7 is the formulation of Embodiment No. 6, wherein the carrier system comprises a liposome.

Embodiment No. 8 is the formulation of one of Embodiment Nos. 6-7, wherein the carrier system comprises a biodegradable polymer, a hydrogel, or a cyclodextrin.

Embodiment No. 9 is the formulation of one of Embodiment Nos. 6-8, wherein the carrier system comprises a nucleic acid complex.

Embodiment No. 10 is the formulation of one of Embodiment Nos. 6-9, wherein the carrier system comprises a virosome.

Embodiment No. 11 is a method of treating anesthesia-induced neurotoxicity, the method comprising administering a formulation comprising an oligonucleotide selected from the group consisting of an oligonucleotide having one of SEQ ID No.:1 through SEQ ID No.:42 or a variant thereof to a subject, wherein the formulation is administered prior to, concomitant with, subsequent to, or combinations thereof administration of a general anesthetic comprising a fluorinated compound.

Embodiment No. 12 is the method of Embodiment No. 11, wherein the oligonucleotide comprises at least 75% sequence identity to one of SEQ ID No.:1 through SEQ ID No.:42.

Embodiment No. 13 is the method of one of Embodiment Nos. 11-12, wherein the oligonucleotide comprises at least 85% sequence identity to one of SEQ ID No.:1 through SEQ ID No.:42.

Embodiment No. 14 is the method of one of Embodiment Nos. 11-13, wherein the oligonucleotide comprises at least 95% sequence identity to one of SEQ ID No.:1 through SEQ ID No.:42.

Embodiment No. 15 is the method of one of Embodiment Nos. 11-14, wherein the oligonucleotide comprises one of SEQ ID No.:1 through SEQ ID No.:42.

Embodiment No. 16 is the method of one of Embodiment Nos. 11-15, wherein the oligonucleotide is incorporated into a carrier system.

Embodiment No. 17 is the method of Embodiment No. 16, wherein the carrier system comprises a liposome.

Embodiment No. 18 is the method of one of Embodiment Nos. 16-17, wherein the carrier system comprises a biodegradable polymer, a hydrogel, or a cyclodextrin.

Embodiment No. 19 is the method of one of Embodiment Nos. 16-18, wherein the carrier system comprises a nucleic acid complex.

Embodiment No. 20 is the method of one of Embodiment Nos. 16-19, wherein the carrier system comprises a virosome.

In some embodiments, administration of the formulations disclosed herein (particularly, formulations including an oligonucleotide having one or more of SEQ ID No.:1 through SEQ ID No.:42 or a variant thereof) substantially contemporaneously with (for example, shortly before, during, or shortly after) the administration of anesthesia reduce the effect on cognitive function resultant from exposure to such anesthesia, that is, to preserve cognitive function in patients undergoing exposure to this class of clinical drugs. In certain instances, the effects of the exposure to the anesthesia may be compounded, such as where the condition being treated may tend to have some effect on cognitive function. In these instances, the formulations disclosed herein (particularly, formulations including an oligonucleotide having one or more of SEQ ID No.:1 through SEQ ID No.:42 or a variant thereof) may be particularly advantageous.

For example, administration of the formulations disclosed herein (particularly, formulations including an oligonucleotide having one or more of SEQ ID No.:1 through SEQ ID No.:42 or a variant thereof) may be particularly advantageous in the context of pediatric anesthetics.

Additionally or alternatively, administration of the formulations disclosed herein (particularly, formulations including an oligonucleotide having one or more of SEQ ID No.:1 through SEQ ID No.:42 or a variant thereof) may be particularly advantageous in the context of patients suffering or suspected from suffering ischemic events such as strokes or heart attacks, for example, by reducing the volume or severity of penumbral or watershed tissue damage that may result from such events.

Additionally or alternatively, administration of the formulations disclosed herein (particularly, formulations including an oligonucleotide having one or more of SEQ ID No.:1 through SEQ ID No.:42 or a variant thereof) may be particularly advantageous in the context of revascularization procedures of the brain, heart, viscera or extremities. For example, the prophylactic use of the disclosed formulations may preserve transiently-stressed tissues which, as a result of such stresses, could potentially participate in triggering or instituting an apoptotic cascade.

While various embodiments in accordance with the principles disclosed herein have been shown and described above, modifications thereof may be made by one skilled in the art without departing from the spirit and the teachings of the disclosure. The aspects described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above but is defined by the claims which follow that scope including all equivalents of the subject matter of the claims. Each and every claim is incorporated as further disclosure into the specification and the claims are embodiment(s) of the present presently disclosed subject matter. Furthermore, any advantages and features described above may relate to specific embodiments but shall not limit the application of such issued claims to processes and structures accomplishing any or all of the above advantages or having any or all of the above features.

Additionally, any section headings used herein are provided to provide organizational cues. These headings shall not limit or characterize the subject matter set out in any claims that may issue from this disclosure. Specifically, and by way of example, although the headings might refer to a “Field,” the claims should not be limited by the language chosen under this heading to describe the so-called field. Further, a description of a technology in the “Background” is not to be construed as an admission that certain technology is prior art to any subject matter of this disclosure. Neither is the “Summary” to be considered as a limiting characterization of the subject matter set forth in issued claims. In all instances, the scope of the claims shall be considered on their own merits in light of this disclosure but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several aspects have been provided in the present disclosure, it should be understood that the disclosed aspects may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1. A formulation comprising an oligonucleotide selected from the group consisting of an oligonucleotide having one of SEQ ID No.:1 through SEQ ID No.:42 or a variant thereof.

2. The formulation of claim 1, wherein the oligonucleotide comprises at least 75% sequence identity to one of SEQ ID No.:1 through SEQ ID No.:42.

3. The formulation of claim 1, wherein the oligonucleotide comprises at least 85% sequence identity to one of SEQ ID No.:1 through SEQ ID No.:42.

4. The formulation of claim 1, wherein the oligonucleotide comprises at least 95% sequence identity to one of SEQ ID No.:1 through SEQ ID No.:42.

5. The formulation of claim 1, wherein the oligonucleotide comprises one of SEQ ID No.:1 through SEQ ID No.:42.

6. The formulation of claim 1, wherein the oligonucleotide is incorporated into a carrier system.

7. The formulation of claim 6, wherein the carrier system comprises a liposome.

8. The formulation of claim 6, wherein the carrier system comprises a biodegradable polymer, a hydrogel, or a cyclodextrin.

9. The formulation of claim 6, wherein the carrier system comprises a nucleic acid complex.

10. The formulation of claim 6, wherein the carrier system comprises a virosome.

11. A method of treating anesthesia-induced neurotoxicity, the method comprising:
administering a formulation comprising an oligonucleotide selected from the group consisting of an oligonucleotide having one of SEQ ID No.:1 through SEQ ID No.:42 or a variant thereof to a subject, wherein the formulation is administered prior to, concomitant with, subsequent to, or combinations thereof administration of a general anesthetic comprising a fluorinated compound.

12. The method of claim 11, wherein the oligonucleotide comprises at least 75% sequence identity to one of SEQ ID No.:1 through SEQ ID No.:42.

13. The method of claim 11, wherein the oligonucleotide comprises at least 85% sequence identity to one of SEQ ID No.:1 through SEQ ID No.:42.

14. The method of claim 11, wherein the oligonucleotide comprises at least 95% sequence identity to one of SEQ ID No.:1 through SEQ ID No.:42.

15. The method of claim 11, wherein the oligonucleotide comprises one of SEQ ID No.:1 through SEQ ID No.:42.

16. The method of claim 11, wherein the oligonucleotide is incorporated into a carrier system.

17. The method of claim 16, wherein the carrier system comprises a liposome.

18. The method of claim 16, wherein the carrier system comprises a biodegradable polymer, a hydrogel, or a cyclodextrin.

19. The method of claim 16, wherein the carrier system comprises a nucleic acid complex.

20. The method of claim 16, wherein the carrier system comprises a virosome.
